(19) United States
(12) Patent Application Publication
(10) Pub. No.: US 2008/0207600 A1
(43) Pub. Date: Aug. 28, 2008

(54) Pharmaceutical Combination for the Treatment of Cns Functional Disorders

(76) Inventors: Naum Isaakovich Goldstein, Berlin (DE); Roman Naumovich Goldstein, Berlin (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/579,208

(22) PCT Filed: Apr. 28, 2005
(86) PCT No.: PCT/RU2005/000233

§ 371 Date: Dec. 21, 2007

(30) Foreign Application Priority Data
Apr. 30, 2004 (RU) 2004113408

(51) Int. Cl. A61K 31/55; A61K 31/195; A61K 31/405; A61K 31/205; A61K 31/502; A61K 31/451; A61P 25/00

(52) U.S. Cl. 514/221; 514/556; 514/563; 514/415; 514/250; 514/327

(57) ABSTRACT

The invention relates to a method and a pharmaceutical combination for the treatment of CNS disorders, which includes at least a first compound having therapeutic effect on the CNS, and a second compound which facilitates penetration through the hematoencephalic barrier of the former. The second compound is administered endonasally, and has refectory (mainly neuro- and vasoactive) effects over structures and receptors of nasal mucous membrane, mainly receptors of vomeronasal systems and trifacial nerve.

### TECHNICAL FIELD

The invention relates to the field of pharmacology and refers to pharmaceuticals which have effect over the central nervous system—specifically, to pharmaceutical combinations usable to control CNS functional disorders, such as extrapyramidal disorders and other diseases of the central nervous system and endocrine system, including drug-controllable extrapyramidal disorders, traumatic hyperkinesias, encephalopathies of various genesis, drug addictions, disseminated sclerosis, cerebral blood circulation disorders, psychotic states, prevention of intrauteral fetal hypoxia, infant cerebral palsies and control over systemic disorders suffered by mothers in the pre- and postnatal period, as well as disorders in adaptation mechanisms and in cerebral neurotransmitter system disbalances.

### BACKGROUND ART

It is known that extrapyramidal disorders are caused by damage to a wide variety of links within cortical-subcortical systems involved in regulation of motor functions. For instance, chorea is usually associated with damage to the caudate nucleus, while dystonia—with damage to the shell (V. N. Shtok, O. S. Levin, N. V. Fedorova. “Extrapiramidalnye rasstroistva”. (Extrapyramidal disorders). Moscow, “MIA” publishers, 2002). To restore the neurotransmitter balance in such cases used are drugs which increase dopaminergic system activity, and drugs which reduce cholinergic- and glutamatergic system activity (cholinolitics, amantadine).

Neurotransmitter functional disbalances are also typical for a number of hyperkinesias. Neurochemically, hyperkinesias may be triggered both by increased dopaminergic system activity and by reduced activity of cholinergic and/or gamma-aminobutyric acid (GAMA)-ergic system.

Organic damage to various departments of nervous system is typical for chronic disseminated sclerosis growing progressively worse, which causes a wide range of neurological symptoms. Sometimes, it is possible to improve pyramidal disorders by way of intravenous carnitine administration. Discoordination and cerebellar tremor are reduced when long-term glycine treatment is applied in combination with small doses of b-blockers and tricyclic antidepressants and Vitamin B6. In case of any deterioration in intellectual & mnestic functions, intranasal administration of semax is prescribed.

Neurotransmitter system disorders are also typical for formation of drug- (opioid-) addiction and alcoholism. As regards opioid addiction, it is asserted that, irrespective of the origin of any particular opioid (whether natural, synthetic or semi-synthetic), it is always a ligand of specific binding sites and has similar effects over neurophysiological disbalances (Yu. P. Sivolap, V. A. Savchenkov. “Farmakoterapia v Narkologii” (Pharmacotherapy in narcology). Moscow, “Meditsina” Publishers, 2000).

The effects of alcohol over CNS is associated with depressive effects of this narcotic substance over cellular membranes of neurons, with disorders taking place in receptor strictures and in ion channels. Neurotransmitter disorders are most typical for withdrawal syndrome combined with CNS hyperexcitability and prevalence of sympathetic neurotransmitter effects (L. S. Freedman, N. F. Fleming, D. G. Roberts, S. E. Hyman. “Narkologii” (Narcology). Moscow-St. Petersburg, 2000).

Disorder in the system of neurochemical vehicles is also typical for development of disadaptogenic neuroexchange & endocrine diseases and essential hypertension. The clinic picture of vegetative disorders involving endocrine regulation system depends on the extent and nature of each particular dysfunction in the hypothalamus & pituitary gland system. It is also known that hyperactivity of medullar ventrolateral glutamatergic neurons involved in arterial pressure regulation, as well as deficit in central noradrenaline contents, may be contributive to arterial hypertension pathogenesis (M. S. Kushakovsky. “Essentialnaya Gipertenzia” (Essential hypertension). St. Petersburg, “Foliant” Publishers, 2002).

What is specific for treatment of infant cerebral palsy (ICP) and autism, two grave diseases setting off for their course of development in infancy of the patients, is that (as of the date, when the treatment is actually initiated) the young patient is usually largely delayed in development, both in relation to his/her equals in age and in terms of his/her own individual biological development program. To a large extent, this is caused by disbalance between various “ergic” neurotransmitter processes taking place in the limbic system (D. W. M. Bishop, 1993, in: I. A. Skvortsov, N. A. Yermolenko, “Development . . . ” P. 254). Improvement of neurotransmitter balance, restoration of neuron functional activity within the CNS in combination with conventional drug treatment and physiotherapy is, as of today, looked upon as a highly promising line in treatment of this infant-age pathology (I. A. Skvortsov, N. A. Yermolenko. “Razvitie nervnoi sistemy u detei v norme n patologii” (Development of nervous system in kids: normal and pathological course). Moscow, “MEDpress-inform” Publishers, 2003).

A special prominence among extrapyramidal pathologies is held by disorders caused by use of a group of drugs capable (apart from their direct beneficial effects upon damaged cerebral structures) to disrupt neurotransmitter balance within structures relatively intact. For instance, neuroleptics blocking dopamine receptors (haloperidol), lithium-based preparations reducing sensitivity of post-synaptic membrane to dopamine, or the sympatholytics depleting dopamine- and serotonine stores in central synapses (reserpine), —all of them may trigger development of extrapyramidal disorders. Prompt elimination or alleviation of side effects caused by the use of a number of medicines is actually impossible at present with the help of existing medicinal preparations.

Drug-based therapy of extrapyramidal diseases is intended to modulate activity of dopaminergic, adrenergic and cholinergic systems in various combinations (Leese A., Ticky J. Translated from English. M. “Meditsina” Publishers, 1989, p. 336).

To control diseases, for which scarcity of movements, muscular rigidity and hypokinesia are typical, prescribed are the drugs activating dopaminergic transmission (levodopas, direct agonists of dopamine receptors, such as parlodel) and inhibiting cholinergic transmission (various drugs of the central M-cholinergic group). To control diseases, for which excessive movements (a wide range of hyperkinesias) are typical, drugs suppressing dopaminergic transmission (neuroleptics of various groups) are used.

Known in the prior art is a method for treatment of extrapyramidal nervous diseases involving intracranial transtympanic injection of a vestibulotoxic drug. As the latter used are injections of antibiotic named streptomycin, and each injection is to be made at side opposite to the area, where the symptoms are most pronounced. This method is used in a case, when in the position of the patient his/her head is held 30-45 grades below the feet, the pathological symptoms are regressive. (Russian Patent No. 2168997, Class: A 61 K 31/036, 2000).

The disadvantages of the known pharmaceutical means and the CNS functional disorder treatment method are that the method is invasive and that there is a risk of complications caused by toxin administration into the cerebral tissue. Furthermore, this method is relatively difficult to perform; it cannot be practiced at the patient's own home and is relatively expensive.

It should be noted that drug treatment of the afore-listed diseases requires continuous use of the medicinal preparations, and their doses have to be continuously extended. This involves the risk of adaptation and addiction, while the used drug will be less and less efficacious and side effects will grow in numbers and severity.

Known in the prior art is intravenous and intranasal use of neuropeptides (Gozes I. “Neuroprotective peptide drug delivery and development: potential new therapeutics” TRENDS IN NEUROSCIENCE, ELSEVIER, Amsterdam, NL, Bd. 24, Nr 12). The drug Neurotrophin affects the d of aminergic neurons of mesencephalon and the motoneurons of the spinal cord, that is, it has therapeutic effect of its own for the patients having Parkinson's disease. (Gozes, p. 702, lines 18-22). The method makes use of penetration of the medicinal substance into the brain in intravenous administration. Used for this purpose are special carrier means having properties, which stimulate absorption, such as the positively charged liposomes with the bio-adhesive effect in relation to the nasal mucous membrane (Gozes, p. 703, para 2 below). Such a drug for intranasal administration contains heptapeptide ASTN 4-10 in the form of 0.1% solution.

However, the cited results are not based on usage of the active forms of oxygen or other radicals, as it is done in this invention, the corresponding sources do not even mention it.

Known in the prior art is a pharmaceutical means for curing pain, which contains an analgesic component and an oxygen radical (German Patent No. 19 514 522, 1995, International Patent Application WO 96/32120). These prior art sources describe a property of the oxygen radical to exponentiate effects produced by analgesics through activation of antinociceptional central mechanisms. It should be noted that in these prior art technologies the analgetics were administered intraperitoneally only.

However, these prior art sources do not reveal, describe, nor anticipate solution to the problem, as stated in this Application, because the patent contains no consideration of new data obtained by the Applicant lately and pertaining to new properties of active oxygen forms explored by the Applicant, including low-molecular radical compounds, such as NO- and CO-active products. As the Applicant's studies revealed, active forms of oxygen in the form for nasal administration may be successfully used as compounds which facilitate to drug and/or metabolite penetration of the hematoencephalic barrier and, therefore, increase availability of drugs for the CNS.

Known in the prior art is a drug used against Parkinson's disease and other tremor-involving diseases. This drug contains oxygen anion-radicals and, as such, water solution of low-concentration hydrogen peroxide (Eurasian Patent No. 001107, Int. Class: A61 K 33/40, 1997 and patent DE 197 08 643 A). These sources describe use of oxygen anion-radicals for treatment of Parkinson's disease and other tremor-involving diseases.

The prior art includes a method of medicinal influence on the patient's organism in treatment of Parkinson's disease involving use of a medicinal means containing water solution of low-concentration hydrogen peroxide to be endonasally administered along with the pharmacological stabilizer (Russian Patent No. 2213565, Int. Cl. A 61 K 33/40, 2002).

The afore mentioned technologies were developed by the inventor of the present invention; these information sources contain description of the ability of oxygen anion-radicals, such as low-concentration hydrogen peroxide, to have therapeutic effects in treatment of Parkinson's disease, which was also discovered by the inventor of the present invention.

However, these prior art sources and all other sources available to the Applicant neither anticipate n or describe the discovered ability of the endonasally used substances, containing active oxygen forms, such as hydrogen superoxide and/or peroxide and/or NO- and/or CO-active products, to facilitate penetration of the hematoencephalic barrier by medicinal preparations and/or metabolites and, thereby, increase their availability for the CNS, which ability manifests itself in the pharmaceutical combination proposed by the applicant.

### BRIEF DISCLOSURE OF THE INVENTION

As shown by the studies of the Applicant, substances usable endonasally, while having refectory (predominantly, neuro- and vasoactive) effects over structures and receptors of nasal mucous membrane (receptors of vomeronasal systems and trifacial nerve, mainly) can modify penetrability of hematoencephalic barrier membrane structures (in the hypothalamic area predominantly) for biologically active substances of various nature, including medicinal preparations, endogenous metabolites and biological additives.

There are known pharmaceutical preparations, such as dimethyl sulphoxide, which can rapidly penetrate through biological membranes, including the skin and mucous membranes. (Entsiklopedia Lekarstv (“Entsiklopedia lekarstv” (Drug Encyclopedia), Issue No. 11, RLS-2004, p. 299). In most cases, this drug is used in combination with other medicinal preparations, in certain pharmaceutical combinations, for example with heparin for treatment of rheumatoid arthritis, or with antibiotics (for treatment of streptodermias, with the drug pre-applied to the skin).

This pharmaceutical combination and the treatment method are most close to the claimed ones.

The disadvantages of the known pharmaceutical combination and treatment method are: the impossibility of their use for treatment of CNS disorders; side effects suffered by nasal mucous membrane and by skin in the form of contact-induced dermatitis, erythemas, burning pains and itching; ocular side effects, such as glaucoma and cataract.

It is the object of this invention is to create an efficacious pharmaceutical combination, containing the main pharmacologically active substances in concentration and dosage lower than those usually administered, and producing as few side effects as possible; development of a method for correction of state connected with C NS functional disorders and involving the proposed pharmaceutical combination; creation of a set of pharmaceuticals containing the developed pharmacological combination; identification of new application for active forms of oxygen and creation of a pharmaceutical means facilitating penetration of the hematoencephalic barrier by medicinal substances and/or metabolites.

The technical result of the proposed inventions is in rapid development and improvement efficacy of the therapeutic treatment, added by lower dosage and alleviated peripheral side effects produced by the drugs, as well as simplicity in practice and simplicity in technological implementation of the applicable method.

Use of this invention allows to improve efficacy of treatment and to extend the range of the therapeutic effects through a better availability for the CNS of drugs, metabolites of artificial origin, and other biologically active substances, thus facilitating restoration of the disrupted neurotransmitter balance between various links of the cortical-subcortical functional complex in treatment of extrapyramidal disorders, depressions and other nervous diseases, including drug-treatable extrapyramidal disorders, traumatic hyperkinesias, encephalopathies of various genesis, drug addictions, disseminated sclerosis, disorders in cerebral blood circulation and adaptation mechanisms, psychotic states, prevention of intrauteral fetal hypoxia, infant cerebral palsies and control over systemic disorders in mothers in the pre- and postnatal period.

Advantages of the claimed invention, as compared to the most close analogue, are: better availability of drugs, metabolites of artificial origin and other biologically active substances for cerebral tissues, including better conditions and higher speed of penetration of the substances into the brain, as well as better efficacy of the brain-penetrating substances through lowering of sensitivity thresholds and facilitation of passage of impulses within brain structures. Furthermore, the claimed pharmaceutical combination contains substances which are important for protection of nasal mucous membrane and for improvement of consumer properties of the combination, such as triglycerides, sodium monohydrogenphosphate, ethereal oils, vegetable extracts and aromatizers.

An important advantage of the claimed pharmaceutical combination is absence of any adverse systemic or metabolic effects caused by the use of the endonasal drugs (for reason of the vanishingly small concentrations of such drugs), as well as a considerable alleviation of side effects caused by the medicines and other biologically active substances, which is caused by considerable decrease in the dosage of such drugs and substances.

### DETAILED DISCLOSURE OF THE INVENTION

The afore-stated objective is attainable through creation of a pharmaceutical combination having effect over functioning of the CNS, containing at least one compound having therapeutic effect on the CNS, and a compound which improves availability of the former compound for the CNS through bringing about better conditions for penetration of the hematoencephalic barrier by the former compound; as the compound facilitating penetration of the hematoencephalic barrier the pharmaceutical combination comprises an endonasally administered means, which has refectory (mainly neuro- and vasoactive) effects over structures and receptors of nasal mucous membrane (mainly structures and receptors of vomeronasal systems and trifacial nerve).

As compounds which have therapeutic effects over the CNS, used could be compounds of the cerebral-neuromediator metabolic predecessor group (for instance, predecessors of dopamine or serotonine, such as levodopa or triptophane); those of the cerebral neurotransmitter system receptor agonist group (such as bromocriptine or lisuride); those of the cerebral neurotransmitter system receptor antagonist group (such as haloperidol or biperiden); those of the psycho- or neurotropic group (such as tramadol, amitriptilyne or diazepam); those of the anabolic, antioxidant or antihypoxant group (such as levocarnitine, methyluracil or mexidol); those of the nootropic group (such as pyracetam), all of which, however, does not put any limits upon the invention.

The former and the latter compounds may be administered simultaneously or one after the other. For instance, within pharmacological combination containing non-prolonged levodopa preparations, such as nacom, synemet or madopar, substances which improve availability of drugs for the CNS are administered either simultaneously or at a small interval, as related to administration of the medicinal substances proper.

In contrast, within pharmacological combination containing other levodopa preparations, such as rapid-effect dispersible madopar-125 tablets, the substance facilitating penetrability of the hematoencephalic barrier is used only after administration of the therapeutic substance, which will allow to improve efficacy and to extend the effective period of the latter at the latest stages of absorption and metabolism.

The compound, having therapeutic effect for the CNS, is mainly for oral administration, which ensures simplicity of the method, for example when taken are levodopa preparations, such as nacom and synemet, or a number of other neurotropic drugs, such as tramadol, amitriptilyne or diazepam. However, parenteral administration, including intravenous, intramuscular, sublingual or on-skin use, is also possible.

As the means for endonasal administration in the proposed pharmaceutical combination used are neuro- and vasoactive substances, which are active forms of oxygen (such as superoxide O^{2.-}) and/or hydrogen peroxide H₂O₂, and/or forms of NO-, and/or CO-active products. For these purposes, single-administration doses of neuro- and vasoactive substances are between 1.0*10⁻¹⁵ g and 1.0*10⁻⁵ g.

Since the means for endonasal administration are non-stable products, the pharmaceutical combination contains a number of pharmacologically acceptable stabilizers. Benzoic acid (or its salts) and ethylenediaminetetraacetic acid (or its salts) may be used as such stabilizers.

Furthermore, since the drug for endonasal administration can have adverse effect over the mucous membrane, which usually manifests itself as an irritation in the mucous membrane, the drug contains substances which protect nasal mucous membrane, such as mannitol, monohydrogenphosphate, triglycerides, ethereal oils and vegetable extracts.

The objective is also achieved by development of a method of correction of states, connected with the CNS functional disorders, wherein the proposed pharmaceutical combination is used, with the drugs administered simultaneously or one after another.

The method is implemented as follows: the patient receives (orally or parenterally) a drug containing, at least, one compound having therapeutic effect for the CNS and the means for endonasal administration, and (depending on pharmacokinetic nature of the former compound), the latter may be administered simultaneously with, before, or after administration of the former.

This invention is intended to protect the set of pharmaceuticals, characterized in that it contains the pharmaceutical combination having effect on the CNS functioning, the said set includes at least one compound therapeutic for the CNS and the means for endonasal administration.

The pharmaceutical set is a set of substances containing at least one compound therapeutic for the CNS and the means for endonasal administration acceptable for use with the former, the said means being in the form for nasal administration (as a spray, in a vial of drops, or as an ointment) and in a complete set with auxiliary devices, such as dropper, spray batcher or pumping seal, as well as a manual for the proper use of the complete set.

Apart from this, the Applicant hereby protects a new property of oxygen active forms, such as, superoxide, and/or hydrogen peroxide, and/or NO-, and/or CO-active products, usable endonasally, namely, the ability to facilitate penetration of the hematoencephalic barrier by medicinal substances and/or metabolites.

This invention also covers the pharmaceutical means for endonasal administration, contained within the proposed pharmaceutical combination, which facilitates penetration of the hematoencephalic barrier by medicinal substances and/or metabolites, the said means contains active oxygen forms such as superoxide, and/or hydrogen peroxide, and/or NO-, and/or CO-active products.

The examples given below are illustrative of the essence of the invention, but they do not limit its scope.

### 1. EXPERIMENTAL EXAMPLES

### Studies Performed Upon Animals

### Example 1.1

### Intensification of the Protective Effect Against Damage Produced by Hyperbaric Oxygen (HBO)

For extending opportunities of the existing drug-based methods of increasing resistance against damage produced by HBO, nasal applications of low-dosage hydrogen peroxide pharmacologically combined with eleuterococus alcoholic solution were used. The study was performed on 38 puberty-age male mice, body mass: 20±2 g. Efficacy of the protective effect was compared with efficacy of eleuterococus as such (Control Group 2) and with results obtained in the technical control (Control Group 1). Animals, which received hydrogen peroxide low-concentration solution pharmacologically combined with eleuterococus, were assigned into the experimental group (Group 0).

Conditions, within which HBO was producing its effects: 3 atm. of pure O₂, dP/dt=0.3 atm*min⁻¹. Animals of the experimental Group 0, before exposure to HBO, received nasal applications of hydrogen peroxide pharmacologically combined with eleuterococus intragastrically (i/gas., through gavage, 0.2 ml of eleuterococus solution based on 30% alcohol pharmacopeal tincture and dissolved in the physiological solution & alcohol mixture). Animals of Group 2 (control group) received, through gavage, 0.2 ml of a similar eleuterococus solution during two weeks before exposure to HBO. Animals of Group 1 (control group) received, in the similar conditions, a mixture of the physiological solution and alcohol. The obtained results were processed statistically, and they are shown in Table 1.1 as M±σ.

Results of the experimental studies demonstrated efficacy of nasal applications of low-dosage hydrogen peroxide for intensification of the protective effect of the eleuterococus-based drug in the external conditions of combined exposure of the organism to physical and chemical factors (high gas pressure and high concentration of oxygen, which is a powerful oxidant).

### Example 1.2

### Intensification of the Pharmacological Effects of Neuroleptic Named Haloperidol

In animals involved in the experimental studies (rats), effective doses of neuroleptic drug named haloperidol produce depression of spontaneous motion activity (SMA). The presently-known clinical methods of haloperidol administration are: intravenous, intramuscular and oral. Although haloperidol does have a limited ability to penetrate the hematoencephalic barrier (HEB), haloperidol is proposed to be used in combination with compound, facilitating penetration of HEB by haloperidol in order to improve haloperidol's therapeutic efficacy. In theory, it is possible to improve the efficacy of the medicine, to reduce the single-administration dosage (through elimination of the effect of “first passage” through the liver) and to alleviate adverse somatic side effects.

The Applicant used pharmacological combination of intraperitoneally (i/p.) administered haloperidol with nasally applied CO-generating composition. It was shown in the preliminary studies that haloperidol-dependant depression in motive responses of the animals develops, on the average, 4.2±1.8 minutes after intraperitoneal administration (dosage: 0.2 mg/kg) and persists for 53.5±7.7 minutes on the average.

The experiments were performed on 24 puberty-age white rats (of no particular breed), body mass: 100-120 grams. Depression in SMA of the animals, as produced by i/p. drug administration, was compared with SMA depression produced by i/p. administration combined with nasal administration of CO-generating composition on the basis of a hem-oxygenase enzyme form. SMA of each animal was evaluated as total sum of values obtained within 2 minutes of “open field” test (in terms of the number of poles/supports, number of ring exercises and cleanings), prior to drug administration, and 10 minutes after drug administration.

The experimental animals were subdivided into 4 groups:

Group No. 1: controls (6 animals), no exposure;

Group No. 2: haloperidol (7 animals), i/p., 0.2 mg/kg;

Group No. 3: haloperidol (5 animals), i/p., 0.05 mg/kg;

Group No. 4: haloperidol (6 animals), i/p., 0.05 mg/kg, in pharmacological combination with CO-generating composition.

Haloperidol was administered by i/p. route within the physiological solution; CO-generating composition was administered nasally. The obtained results were statistically processed, and they are shown in Table 1.2 as M±σ.

The experiments proved better efficacy of haloperidol if administered by i/p. route in pharmacological combination with nasal application of CO-generating composition. Intensification of the effects of haloperidol is attainable through bringing about a better HEB penetrability and increasing the sensitivity of cerebral receptor structures to the drug.

### 2. EXAMPLES OF CLINICAL TESTS PARTICIPATED IN BY VOLUNTEERS

2.1. Use of the pharmaceutical combination in depressed patients.

This study involving 9 outpatients suffering highly pronounced depression was based on the use of pharmaceutical combination of an endonasal drug, as based on hydrogen peroxide or the source of glycerolnitrate NO-form, and oral administration of antidepressants and/or tranquilizers.

All patients had alleviation or complete elimination of all clinical signs of depression and general shift of their prevalent moods up to higher scales. There was alleviation of headaches and somatic pains and of actual anxieties. Night-time sleep quality was improved. Day-time activity rates increased. All this allowed to reduce daily dosage of antidepressants and tranquilizers by 50-70% in patients treated with these drugs.

2.2. Use of the pharmaceutical combination in drug-based treatment of extrapyramidal disorders and encephalopathies.

The clinical studies were performed upon 30 patients, of whom 4 were diagnosed as having “endogenous phychotic state”, 13—as having “discirculatory encephalopathy on the background of brain atherosclerosis”, 1—as having complex-genesis encephalopathy on the background of CNS intoxication, 12—as having “cerebral atherosclerosis with mental disorders”. Disease suffering period: 4-11 years. All patients received drugs usually prescribed for treatment of corresponding diseases: alprazolam, betahistine, synemet, nacom, madopar umex, midanthane, triphen—in various dosage.

Average time of treatment by the claimed pharmaceutical combination of drugs with the use of compounds, facilitating penetration of HTB by drugs, made up 3 weeks. In each patient positive results were achieved. For instance, hyperkinesias were significantly alleviated in patients with posttraumatic syndrome. Depression was also alleviated, and generally prevalent moods were improved.

These results were obtained with average daily drug dosage reduced by 30-50%. Improvements were observed in all cases, and in 11 patients these improvements were considerable. The major part of the patients reported better prevalent moods, a good vivacity, higher activity rates and considerably lower depression levels. Treatment with the pharmaceutical combination brought about an essential extension of opportunities available for patients in growing less dependent upon daily care provided to them by others.

2.3. Use of the Pharmaceutical Combination in Patients with Essential Hypertension.

The studies were performed upon 14 patients, age group: 65-75 years, concomitant diagnosis: “Hypertensive disease”, arterial pressure (AP): 170-180/90-100 mercury. Upon completion of 50-60 day treatment course with the use of the pharmaceutical combination, 12 patients had their AP reduced (normalized) down to 130-140/85-90 mercury, with daily dosage of in-use antihypertensive drugs reduced by 30% on the average. The achieved positive clinical effect was maintained for 30-45 days after discontinuation of administration of nasal forms increasing HEB penetrability.

2.4. Use of the Pharmaceutical Combination in Patients with Opiate Addiction.

Several short-term treatment courses were performed upon 7 male patients with opiate addiction and currently suffering withdrawal syndrome, namely: 24-48 hours after the latest taking of the abused drug. Symptoms of withdrawal syndrome in such patients included such objectively observable phenomena as tremor, pyloerection, tachicardia, lacrimation, rhinorrhea, fever, etc. Personally, the patients reported muscular pains, chills, headaches, anxiety. The treatment involved, mainly, repeated use of nasal applications until achievable alleviation in manifestations of withdrawal syndrome, in pharmaceutical combination with single-time injection of the habitually-taken drug of abuse within dosage equal to about ¼-⅙ of the usually taken single-time dose, and/or conventional medicine-based treatment involving use of methadone, tramadol and other medicines.

The obtained positive results were manifested in reduction and/or elimination of the most part of symptoms of withdrawal syndrome. This condition was maintained for the period stretching from 24 hours after the first use of the treatment procedures to 72 hours after the third nasal application.

2.5. Use of the Pharmaceutical Combination in Alcoholic Patients.

Several short-term treatment courses were performed upon 6 male patients suffering alcoholic hangover syndrome. Symptoms of withdrawal syndrome in such patients were: tremor, nausea and/or vomiting, psychomotor agitation and vegetative hyperactivity, hyperreflexion, headaches, etc. The treatment involved, mainly, repeated use of nasal applications in pharmaceutical combination with sublingual administration of glycine and/or biotredine and oral administration of benzodiazepines in dosage equal to about ¼ of the conventional dosage.

The obtained positive results were: reduction and/or elimination of the most part of symptoms of alcoholic hangover syndrome. The improvement usually developed, on the average, 15-30 minutes after use of the pharmaceutical drug combination.

2.6. Use of the Pharmaceutical Combination for Symptomatic Treatment of Disseminated Sclerosis.

Clinical observations of symptomatic improvements in patients diagnosed as having “disseminated sclerosis” were performed in five cases. Description of one of the typical cases is stated below.

A female patient of 38 years of age, diagnosis: “disseminated sclerosis”, cerebrospinal form, progredient course. Disease period: 8 years. Clinically, the patient suffered spastic paresis of lower limbs, high-scale reflexes upon her feet and hands, pathological signs upon her feet and hands. Within the latest years, the patient could not move without aid and had to live confined to bed. She had been usually taking the following neurotropic drugs: Memantine™ (10 mg/day) and Midocalm™ (300 mg/day).

This was the prevalent state of the patient, when initiated was treatment with pharmaceutical combination containing the Parcon™ drug based on hydrogen peroxide (three nasal applications daily), which is the product developed by the Applicant and manufactured in proper product batches, and with drugs having predominantly central neuroprotective and antispastic effects. Two weeks later, a tonal normalization (spasticity alleviation) in skeletal muscles was observed, as well as memory improvement and depression alleviation; the patient showed an increased interest towards more active way of living. Three weeks after the treatment was initiated, the patient was able to walk without any aid from others. The patient grew more independent in terms of daily taking care of herself.

Therefore, advantages of the claimed invention are: greater accessibility of drugs, metabolites of artificial origin, and other biologically active substances for cerebral tissues, including better conditions and higher speed of brain penetration by the under-consideration substances, as well as greater efficacy of the brain-penetrating substances through reduction of sensitivity thresholds and facilitation of impulse passage within brain structures.

An important advantage of the claimed pharmaceutical combination is absence of any adverse systemic and metabolic side effects, such as may be caused by use of endonasal drugs (for reason of the vanishingly small concentrations of such drugs) and considerable alleviation of side effects produced by drugs and other biologically active substances (for reason of considerable reduction in dosage of such drugs and substances).

TABLE 1.1
Improvement of mice survival rate exposed to HBO through use of
hydrogen peroxide nasal applications (concentration:
10⁻⁵ mole/liter) in pharmacological combination with
i/gas. administration of eleuterococus.

| | | Data |
|---|---|---|
| Animal group | Survival rate, % | reliability (P) |
| Group 1 (control group): | | |
| technical control; n = 14 | 49.6 ± 9.1 | — |
| Group 2 (control group): | | |
| i/gas, administration of eleuterococus | 56.2 ± 8.2 | 0.1 (2 vs. 1) |
| solution; n = 12 | | |
| Group 0 (experimental group): | | |
| nasal administration of low-dosage | 98.8 ± 8.8 | <0.05 (3 vs. 2) |
| hydrogen peroxide in | | <0.01 (3 vs. 1) |
| pharmacological combination with | | |
| eleuterococus; n = 12 | | |

TABLE 1.2
Comparative data on haloperidol efficacy as shown in spontaneous
activity displayed by under-study animals (rats) in “open field”
tests (i/p. administration) and in the pharmacological combination
of i/p. administration and nasal application of CO-generating
composition.

| | Spontaneous activity | |
|---|---|---|
| | (total values) | |
| | a) before | b) after |
| | haloperidol | haloperidol |
| Animal group | administration | administration |
| Group No. 1: controls, without | 44 ± 6 | — |
| exposure, n = 6 | | |
| Group 2: haloperidol (i/p., 0.2 mg/kg), | 43 ± 5 | 3 ± 1*⁾ |
| n = 7 | | |
| Group 3: haloperidol (i/p., 0.05 mg/kg), | 42 ± 7 | 24 ± 3**⁾ |
| n = 5 | | |
| Group 4: haloperidol (i/p., 0.05 mg/kg), | 45 ± 5 | 2 ± 2^{#)} |
| in pharmacological | | |
| combination with CO-generating | | |
| composition, n = 6 | | |

*⁾P < 0.01 (Group 2 versus Group 1, 2a);
**⁾P < 0.05 (Group 2b versus Group 2a and 1b);
^{#)}P < 0.01 (Group 2b versus Group 4a and 3b).

1.-22. (canceled)

23. A pharmaceutical combination, comprising
a first compound having therapeutic effect on the CNS, and
a second compound that is a neuro- or a vasoactive substance selected from the group consisting of a superoxide, a peroxide, and NO-, and CO-active products, said second compound being provided in a form suitable for intranasal administration, wherein said second compound facilitates penetration of a hematoencephalic barrier by said first compound.

24. The pharmaceutical combination of claim 23, wherein said second compound has reflectory effects over structures and receptors of nasal mucous membrane and has effects on receptors of vomeronasal systems and/or on trifacial nerve.

25. The pharmaceutical combination of claim 23, wherein said first compound is in a form suitable for oral administration.

26. The pharmaceutical combination of claim 23, wherein said first compound is selected from the group consisting of a nootropic compound, a neurotropic compound, a psychotropic compound and a neurotransmitter precursor.

27. The pharmaceutical combination of claim 26, wherein said first compound is a precursor of dopamine or serotonin.

28. The pharmaceutical combination of claim 23, wherein said first compound is a neurotransmitter receptor agonist or antagonist.

29. The pharmaceutical combination of claim 28, wherein said neurotransmitter receptor is a dopamine or serotonin receptor.

30. The pharmaceutical combination of claim 29, wherein said first compound is an antagonist of an acetylcholine receptor.

31. The pharmaceutical combination of claim 23, wherein said first compound is a modulator of GABA-ergic processes.

32. The pharmaceutical combination of claim 23, wherein the molar ratio of the first and the second compounds is between 1:1 and 1:1×10⁻¹².

33. The pharmaceutical combination of claim 23, further comprising a pharmacological stabilizer.

34. The pharmaceutical combination of claim 33, wherein said stabilizer comprises benzoic acid, or its derivatives, or salts of ethylenediaminetetraacetic acid.

35. The pharmaceutical combination of claim 23, further comprising a substance which protects nasal mucous membranes.

36. The pharmaceutical combination of claim 35, wherein said substance which protects nasal mucous membranes is selected from the group consisting of mannitol, triglycerides, monohydrogenphosphate, ethereal oils and vegetable extracts.

37. The pharmaceutical combination of claim 23, wherein said second compound is in the form of nasal spray, drops or ointment.

38. A method of treatment of a CNS functional disorder comprising administering the pharmaceutical combination of claim 23 to a patient having said CNS functional disorder, wherein said second compound is administered intranasally.

39. The method of claim 38, wherein said first compound is administered orally.

40. The method of claim 38, further comprising administering a pharmacological stabilizer.

41. The method of claim 40, wherein said stabilizer comprises benzoic acid, or its derivatives, or salts of ethylenediaminetetraacetic acid.

42. The method of claim 38, further comprising administering a substance which protects nasal mucous membranes.

43. The method of claim 42, wherein said substance which protects nasal mucous membranes is selected from the group consisting of mannitol, triglycerides, monohydrogenphosphate, ethereal oils and vegetable extracts.
